(19) United States
(12) Patent Application Publication
(10) Pub. No.: US 2009/0053314 A1
(43) Pub. Date: Feb. 26, 2009

(54) SUBMICRONIZATION OF PROTEINS USING SUPERCRITICAL FLUIDS

(75) Inventors: Dong Jin PYO, Kangwon-do (KR); Dahl Kyun OH, Kangwon-do (KR)

Correspondence Address: THE RAFFERTY PATENT LAW FIRM, 1952 Gallows Road, Suite 200, Vienna, VA, 22182-3823, US

(73) Assignee: REGERON, INC., Kangwon-do (KR)

(21) Appl. No.: 11/908,893

(22) PCT Filed: Mar. 10, 2006
(86) PCT No.: PCT/KR2006/001051

§ 371 Date: Sep. 17, 2007

(30) Foreign Application Priority Data
Mar. 22, 2005 (KR) 10-2005-0023618
Dec. 16, 2005 (KR) 10-2005-0124456

(51) Int. Cl. A61K 38/22; F26B 21/06; A61K 9/14

(52) U.S. Cl. 424/489; 514/12; 34/83

(57) ABSTRACT

An apparatus and a method for the submicronization of proteins using supercritical fluids is provided for feeding the supercritical fluid and feeding a protein solution using a precipitation vessel with a taper shape at its lower part, in which the precipitation vessel accommodates the supercritical fluid and the protein solution to generate submicroparticles of the protein, and a spray nozzle with coaxial arrangement having an outer nozzle for spraying the supercritical fluid and an inner nozzle for spraying the protein solution.

### FIELD OF THE INVENTION

The present invention relates to an apparatus and a method for the submicronization of proteins using supercritical fluids.

### DESCRIPTION OF THE RELATED ART

Generally, protein drugs exhibit low absorption efficiency to gastrointestinal tract due to their higher molecular weight compared to chemical drugs and natural product drugs. Furthermore, protein drugs have delivery shortcomings in that they are inactivated and decomposed by stomach acid and proteases, and cleared by immune system. Therefore, most protein drugs are administered in the form of injections. However, the injections are inconvenient in view of administration, storage and transportation. In addition, patients' compliance with injections is much lower. Therefore, numerous research efforts to develop a new delivery system for protein drugs have been made in order to conveniently administer protein drugs through methods other than injection without sacrificing the bioavailability of protein drugs. Among them, administration approaches via the lungs and nasopharynx have been suggested. Because administration via the nasopharynx shows relatively low absorption efficiency, its application to protein drugs may be limited. Hussain, A. A., has reported that a low proportion of administered proteins can be directly absorbed into neuronal tissues via mucosal membrane of nose (Hussain, A. A., Adv. Drug. Delivery Rew. 1998, 29-39).

Transpulmonary formulations for administration via the lungs has become the most prominent in the delivery of protein drugs. The pulmonary alveolus with very large surface area has a shorter distance to blood vessels due to its thin epidermis, and shows low metabolic activity and well-developed capillary blood vessels, which provides an environment efficient for absorbing proteins. Therefore, drug delivery via the lungs is promising for the administration of macromolecules such as proteins. While oral-administered drugs are absorbed in the stomach and intestines, transpulmonary drugs are anticipated to be satisfactorily absorbed only in pulmonary alveoli. The diameter of drug particles is required to be less than 5 μm to reach pulmonary alveoli via inhalation. Furthermore, to anticipate consistent delivery with high absorption efficiency to pulmonary alveoli, transpulmonary drugs are required to show homogeneous particle size. Therefore, for developing transpulmonary protein drugs, it is necessary to prepare homogeneous particles having a size of less than 5 μm.

Conventional milling and crushing preparations scarcely produce protein particulates. Therefore, an emulsion process using organic solvents has been used for forming protein particulates; however, this process also produces residual organic solvents and massive quantities of wasted organic solvents and water. Furthermore, the application of this process requires development of a mass production process.

The method of produce particulates by extraction using supercritical fluids under high pressure was first suggested by Hanny and Hogarth. Recently, much research has focused on the supercritical fluid technology (Hannay, J. B. Hogarth, J. Proc. Roy. Sec. London. 1879, 29, 324). It could be appreciated that the supercritical fluid technology could produce micron-, submicron-, or even nano-particulates having narrow size distribution with no residual organic solvents. In addition, this technology may generate nanoscale particulates.

However, there has been no practical case of successfully generating homogeneously nanoscale particulates of proteins using supercritical fluids.

Throughout this application, several patents and publications are referenced and citations are provided in parentheses. The disclosure of these patents and publications is incorporated into this application in order to more fully describe this invention and the state of the art to which this invention pertains.

### BRIEF DESCRIPTION OF THE DRAWINGS

FIG. 1a schematically represents one embodiment of the present apparatus for the submicronization of proteins. The numbers, 1, 2, 3, 4 and 5 denote a means for feeding the supercritical fluid, a means for feeding a protein solution, a precipitation device, a particle collection device and an effluent collection device, respectively. The numbers 11, 12 and 13 denote a carbon dioxide cylinder, a cooling circulator and a high pressure liquid pump, respectively. The numbers 21, 22 and 23 denote a protein solution container, a HPLC pump and a valve. The numbers 31, 32, 33, 34, 35 and 36 denote a heat exchanger, an oven, a pressure gauge, a back pressure regulator, a coaxial nozzle and a precipitation vessel, respectively. The numbers 41, 42, denote a screening device and a particle collector, respectively and numbers 411 and 422 denote a metal frit.

FIG. 1b schematically represents one embodiment of the coaxial nozzle contained in the present submicronization apparatus. The numbers 351 and 352 indicate an outer nozzle and an inner nozzle, respectively.

FIG. 1c schematically represents one embodiment of the precipitation vessel contained in the present submicronization apparatus. The numbers 361 and 362 indicate an upper part and a lower part of the precipitation vessel, respectively.

FIG. 1d schematically represents one embodiment of the screening device contained in the present submicronization apparatus.

FIG. 2a is an FE-SEM image of human growth hormone (hGH) submicroparticles formed according to Experiment 1 in Example III.

FIG. 2b represents the size distribution of human growth hormone (hGH) submicroparticles formed according to Experiment 1 in Example III. The values of y-axis are represented by the ratio of the amount of particles with a predetermined size to the overall amount of particles collected in the particle collection device.

FIG. 2c is an FE-SEM image of hGH submicroparticles formed according to Experiment 3 in Example III.

FIG. 3a is an FE-SEM image of hGH submicroparticles formed according to Experiment 6 in Example IV.

FIG. 3b is an FE-SEM image of hGH submicroparticles formed according to Experiment 8 in Example IV.

FIG. 4 is an FE-SEM image of hGH submicroparticles formed according to Experiment 10 in Example V.

FIGS. 5a and 5b represent HPLC analysis results of hGH submicroparticles (FIG. 5b) and NIBSC standard hGH (FIG. 5a). The x-axis and y-axis indicate retention time (min) and UV absorption intensity, respectively.

FIG. 5c represents analysis results of the biological activity of hGH submicroparticles and NIBSC standard hGH. The x-axis is log values of hGH concentration (ng/ml). The y-axis is the percentage values of the number of cells cultured in hGH-containing medium to the number of cells cultured in control medium [(the number of cells cultured in hGH-containing medium)/(the number of cells cultured in the same medium lacking hGH)×100]. The term “submicronized hGH” used herein refers to hGH particulates formed according to the present invention.

### DETAILED DESCRIPTION OF THIS INVENTION

The present inventors have performed intensive research to provide a novel apparatus and method for the submicronization of proteins enabling the development of transpulmonary and oral-administered formulations of protein drugs. As a result, the present inventors have developed a novel apparatus and method for the submicronization of proteins based on the supercritical fluid process, ensuring the production of nanoscale protein particulates having homogeneous physicochemical properties.

Accordingly, it is an object of this invention to provide an apparatus for the submicronization of a protein. It is another object of this invention to provide a method for the submicronization of a protein. Other objects and advantages of the present invention will become apparent from the detailed description to follow taken in conjugation with the appended claims and drawings.

In one aspect of this invention, there is provided an apparatus for the submicronization of a protein using a supercritical fluid, which includes a means for feeding the supercritical fluid, a means for feeding a protein solution, a precipitation vessel with a taper shape at its lower part, in which the precipitation vessel accommodates the supercritical fluid and the protein solution to generate submicroparticles of the protein, and a spray nozzle with coaxial arrangement having an outer nozzle for spraying the supercritical fluid and an inner nozzle for spraying the protein solution, in which the spray nozzle is connected to the means for feeding the supercritical fluid and the means for feeding a protein solution, in which an outlet end of the inner nozzle protrudes farther than an outlet end of the outer nozzle toward an inner portion of the precipitation vessel, and in which contact between the supercritical fluid and the protein solution occurs in the precipitation vessel.

Proteins submicronized by the present apparatus are not restricted; preferably, they include hormones, hormone analogues, enzymes, enzyme inhibitors, signal transduction proteins or fragments thereof, antibodies or fragments thereof, single chain antibodies, binding proteins or fragments thereof, peptides, antigens, adhesive proteins, structural proteins, regulatory proteins, toxin proteins, cytokines, transcription regulatory proteins, blood clotting proteins and plant defense-inducing proteins. The term used herein “protein” refers to a molecule made up of amino acids jointed together by peptide bonds, e.g., oligopeptide and polypeptide.

Most preferably, the protein submicronization apparatus of this invention is applied to submicronization of human growth hormone.

The apparatus of this invention is designed to embody SEDS (solution enhanced dispersion by supercritical fluids) process among various technologies using supercritical fluids. In the SEDS process, a protein solution and a supercritical fluid are injected and mixed in a precipitation vessel through coaxial nozzles to induce the oversaturation of the protein solution, thereby forming protein microparticles (J. Jung, M. et al., Particle Design using Fluids: Literature and Patent Survey, J. Supercrit. Fluids, 20:179-219 (2001)).

The supercritical fluid used in the present apparatus is preferably selected from the group consisting of carbon dioxide, ethane, ethylene, sulfur hexafluoride, nitrous oxide, chlorotrifluoromethane, monofluoromethane, xenone and combinations thereof, most preferably, carbon dioxide.

The protein solution used in the present apparatus is preferably prepared by dissolving the protein into a solvent selected from the group consisting of water, ethanol, methanol, DMSO (dimethylsulfoxide), isopropanol, acetone, THF (tetrahydrofuran), acetic acid, ethyleneglycol, polyethyleneglycol, N,N-dimethylaniline and combinations thereof. More preferably, the protein solution is prepared by dissolving the protein into a mixture of water and ethanol. Most preferably, the protein solution is prepared using a mixed solution of ethanol and aqueous buffer containing salt (most preferably, NaPO₄) or salt and EDTA.

According to a preferred embodiment, an upper part (361) of the precipitation vessel (36) has a diameter of 1.2-1.5 fold, more preferably, about 1.3 fold larger than the end of the lower part (362) of the precipitation vessel. Such configured precipitation vessel (36) defines an outlet having a streamlined shape, preventing protein particles thus formed from adhering and sticking to the precipitation vessel (36).

According to a preferred embodiment, an upper part (361) of the precipitation vessel (36) has a length of 10-18 fold, more preferably, 13-17 fold, most preferably, about 15.7 fold longer than the lower part (362) of the precipitation vessel (36).

Preferably, the outer nozzle (351) of the spray nozzle (35) with coaxial arrangement has a diameter of 3-6 fold, more preferably, 3-5 fold, most preferably, about 4.3 fold larger than the inner nozzle (352).

According to a preferred embodiment, the outer nozzle (351) has a substantially constant diameter along its overall length. Unlike the outer nozzle, the inner one (352) has preferably a taper shape at its outlet end. In particular, the outlet end of the inner nozzle (352) has a continuously decreased diameter toward the precipitation vessel (36). This taper shape of the inner nozzle (352) allows to greatly increase the difference in pressures before and after spraying the protein solution, which contributes to the decrease in the size of the protein solution droplets and the widening of spray angle. As a result, the taper shape enhances mass transfer effect between droplets of the protein solution and supercritical fluids to form protein particulates with smaller size.

According to a preferred embodiment, an upper part of the inner nozzle (352) has a diameter of 2-4 fold, more preferably, about 3.3 fold larger than the outlet end of the inner nozzle (352).

A striking feature of the present submicronization apparatus is that the outlet end of the inner nozzle (352) is more protruded than the outlet end of the outer nozzle (351) toward the inner portion of the precipitation vessel (36). By such protrusion configuration, the protein spray nozzle (the inner nozzle) is positioned ahead of the supercritical fluid spray nozzle (the other nozzle) and the flow of supercritical fluids, e.g. carbon dioxide, therefore passes through the nozzle before the flow of protein solutions. Such spraying effect of the beforehand carbon dioxide flow gives rise to the rapid jet breakup of protein solutions, causing more effective spraying of protein solutions. As results, proteins solutions are sprayed in smaller droplets and shorter time and the mass exchange effect occurs more effectively, yielding protein particulates having smaller size and homogeneous physicochemical properties. In addition, a chance in collision events between droplets of protein solutions earlier formed is decreased due to the protrusion configuration of the inner nozzle (352) and therefore the form of protein solution droplets is well maintained during mass exchange (i.e., a solvent in a protein solution is diffused into a supercritical fluid and the supercritical fluid is diffused into the protein solution), resulting in the efficient formation of protein particulates with smaller size.

According to a preferred embodiment, the outlet end of the inner nozzle (352) is protruded by 1-10 mm, more preferably, 1-5 mm, still more preferably, 1-3 mm, most preferably, about 1.5 mm toward the inner portion of the precipitation vessel (36) compared to the outlet end of the outer nozzle (351).

Preferably, the apparatus further comprises a particle collection device (4) connected to the precipitation vessel (36). The particle collection device (4) comprises a screening device (41) and a particle collector (42). The present apparatus forms generally nanoscale protein particulates but also forms microscale particulates in small proportion. In order to filter out protein particulates with microsize or over, the screening device (41) is connected to the precipitation vessel (36). The flow of protein particulates passed through the screening device (41) is then introduced into the particle collector (42) where homogenously nanoscale protein particulates are collected. The screening device (41) is equipped with a filter (411, e.g., metal frit) having a predetermined pore size, preferably, 5-40 μm, more preferably, 5-30 μm, still more preferably, 10-25 μm, most preferably, about 20 μm. The particle collector (42) is equipped with a filter (422, e.g., metal frit) having a predetermined pore size, preferably, 0.1-1 μm, more preferably, 0.1-0.6 μm, most preferably, about 0.2 μm.

Solutions having passed through the particle collector (42) are then separated into supercritical fluids and solvents for proteins. The solvents are collected into the effluent collection device (5). The supercritical fluid having passed through particle collector (42) may be recycled or discharged into the atmosphere.

The density of supercritical fluids, e.g., carbon dioxide, increases as their pressure increases and in turn they diffuse more rapidly into a solvent for proteins, e.g., a mixed solution of ethanol and water, thereby causing the oversaturation of a solute in droplets (e.g., human growth hormone) in a faster manner. The rapid oversaturation generates larger amount of crystal nuclei in droplets. However, a high pressure of supercritical fluids may result in negligible change in the size of protein particulates finally formed. This is why protein droplets are sprayed into high pressurized supercritical fluids, e.g. carbon dioxide and in turn their surface area is likely to be decreased, thereby decreasing a surface area for mass exchange. In addition, the high pressure of supercritical fluids induces aggregation between protein particulates formed. Therefore, while the increase in pressure of supercritical fluids promotes the mass exchange between supercritical fluids and solvents, it also exerts the adverse effect on the surface area for mass exchange. Consequently, it could be appreciated that the determination of suitable pressures of supercritical fluids is an important factor for protein submicronization.

According to a preferred embodiment, the pressure of supercritical fluids (particularly, carbon dioxide) ranges from 90 to 130 bar. If the pressure is less than 90 bar, the phase of supercritical fluids is very likely to be broken; in the case of exceeding 130 bar, the structure or conformation of proteins is liable to change. More preferably, the pressure of supercritical fluids (particularly, carbon dioxide) is about 90-100 bar, most preferably, about 90 bar.

In the preparation of protein particulates, the surface tension of sprayed droplets of protein solutions is inversely proportional to temperature. The increase in temperature induces the decrease in the surface tension of droplets, which makes it easier for supercritical fluids to penetrate into droplets. Then, mass exchange between a solvent in droplets and supercritical fluid may be more active. However, extremely high temperature is very likely to elicit aggregation between protein particulates. Furthermore, the increase in temperature under certain pressure results in the decrease in the density of supercritical fluids, to reduce the rate of oversaturation of a solute (e.g., human growth hormone) in droplets, finally forming lager protein particulates. Therefore, it could be understood that the determination of suitable temperature of precipitation vessel (i.e., temperature of supercritical fluids) is one of important factors for protein submicronization.

According to a preferred embodiment, the temperature in the precipitation vessel (36), i.e., temperature of supercritical fluids maintains within 35-45° C., more preferably, 38-42° C., most preferably, about 40° C.

Proteins in droplets form crystal nuclei during mass exchange between a solvent of protein solutions and supercritical fluids. At this time, if the concentration of protein solutions is low, the formation of crystal nuclei occurs slowly and proteins are aggregated around the crystal nuclei to produce larger protein particulates. In contrast, where the concentration of protein solutions is high, numerous crystal nuclei are formed in more rapid manner to be bound each other, thereby decreasing the number of crystal nuclei earlier formed and then increasing the size of final protein particulates. Therefore, it could be recognized that the determination of suitable concentration of protein solutions is another important factor for protein submicronization.

According to a preferred embodiment, the protein solution has a concentration of 10-300 mg/L, more preferably, 20-150 mg/L, still more preferably, 20-35 mg/L, still further more preferably, 20-26 mg/L, most preferably, about 24.4 mg/L.

Preferably, the ratio of a flow rate of the supercritical fluid fed by the means (1) for feeding the supercritical fluid to a flow rate of the protein solution fed by the means (2) for feeding the protein solution ranges from 50:1 to 120:1, more preferably, 75:1-110:1, most preferably, about 100:1.

According to a preferred embodiment, the flow rate of the supercritical fluid fed by the means (1) for feeding the supercritical fluid is 10-40 ml/min, more preferably, 20-30 ml/min, most preferably, about 30 ml/min.

According to a preferred embodiment, the flow rate of the protein solution fed by the means (2) for feeding the protein solution is 0.2-0.8 ml/min, more preferably, 0.3-0.5 ml/min, most preferably, about 0.3 ml/min.

The submicroparticles of proteins formed by the present apparatus have preferably an average diameter of 30-60 nm, more preferably, 35-55 nm, most preferably, about 45 nm.

One of advantages of the present apparatus is to provide homogeneously nanoscale protein submicroparticles. The submicroparticles of proteins formed by the apparatus have a size distribution of no less than 70%, preferably 80%, more preferably 90% within an average diameter of 35-55 nm.

The present apparatus for the submicronization of proteins is very useful in the submicronization of human growth hormone. Preferably, the protein is human growth hormone, the supercritical fluid is carbon dioxide, and the protein solution is prepared by dissolving human growth hormone in a mixed solution of water and ethanol.

Where the present apparatus is applied to the submicronization of human growth hormone, the descriptions for the apparatus for the submicronization of human growth hormone follow those for the apparatus the submicronization of proteins described hereinabove. For example, the setup and configuration of the precipitation vessel (36), the setup and configuration of the outer nozzle (351) and the inner nozzle (352), the pressure and temperature of supercritical fluids, the concentration of protein solutions, the flow rate of supercritical fluids and protein solutions, and the particle size of final products are common between apparatuses for the submicronization of human growth hormone and general proteins.

In another aspect of this invention, there is provided a method for the submicronization of a protein, which includes mixing a protein solution and a supercritical fluid by spray-injecting the protein solution and the supercritical fluid into an inner portion of a precipitation vessel through a spray nozzle with coaxial arrangement, and obtaining submicronized protein particles generated by the precipitation from the protein solution, in which the supercritical fluid is carbon dioxide, the supercritical fluid has a pressure of 90-130 bar, a temperature in the precipitation vessel remains within 35-45° C. and the protein solution has a concentration of 10-300 mg/L.

Since the descriptions for the present method follow those for the apparatus the submicronization of proteins described hereinabove, the common descriptions between them are omitted in order to avoid undue redundancy leading to the complexity of this specification.

For example, the type of proteins applied, solvent in protein solutions, the pressure and temperature of supercritical fluids, the concentration of protein solutions, the flow rate of supercritical fluids and protein solutions, and the particle size of final products are common between them.

According to a preferred embodiment, the protein solution and the supercritical fluid in step (i) are spray-injected through an inner nozzle and an outer nozzle of a spray nozzle with coaxial arrangement, respectively, and an outlet end of the inner nozzle is more protruded than an outlet end of the outer nozzle toward the inner portion of the precipitation vessel.

The present invention provides more homogeneous and smaller protein (particularly, human growth hormone) particulates, permitting the production of transpulmonary drugs of proteins (particularly, human growth hormone). It has been well known that the diameter of drug particles is required to be less than 5 μm to reach pulmonary alveoli via inhalation and to be more than 1 μm to remain in pulmonary alveoli. However, to our best knowledge, the requirement for the diameter of transpulmonary drugs might be erroneous. Practically, the nanoparticles having the size of less than 1 μm could remain in pulmonary alveoli with higher possibility by holding breath for a period of time after inhalation. The nanoparticles of protein drugs increase greatly their delivery efficiency and bioavailability. Where protein drugs are formulated with large particles, they are likely to be eliminated by immune system due to longer period of time for solubilization and to elicit adverse effects by remained particles. In this context, it is very preferable that nanoparticles of protein drugs are administered via lung.

In accordance with the present invention, submicronized protein powder possessing its inherent activities can be prepared with higher concentrations in smaller volumes without using excipients or carriers with the amount generally 15-fold larger than protein active ingredients when preparing protein (particularly, human growth hormone) powder for injection. Therefore, the present invention can enable the development of protein drugs for oral administration.

The following specific examples are intended to be illustrative of the invention and should not be construed as limiting the scope of the invention as defined by the appended claims.

### EXAMPLES

### Example I

### Setting Up of Apparatus for Protein Submicronization

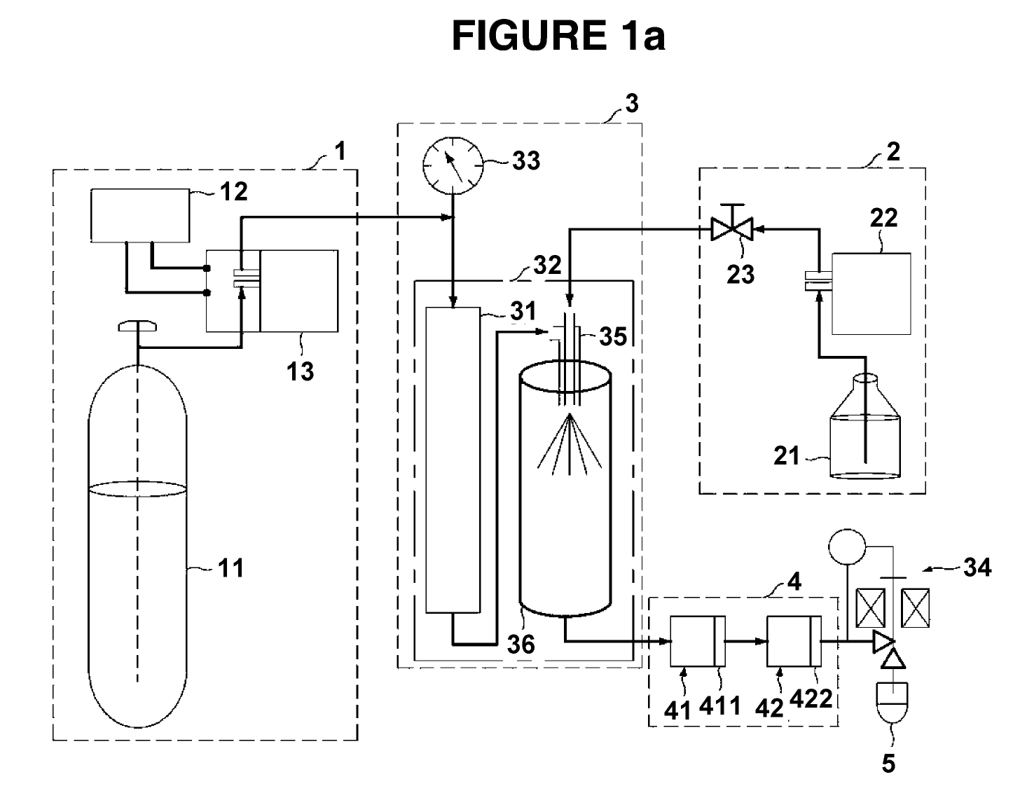

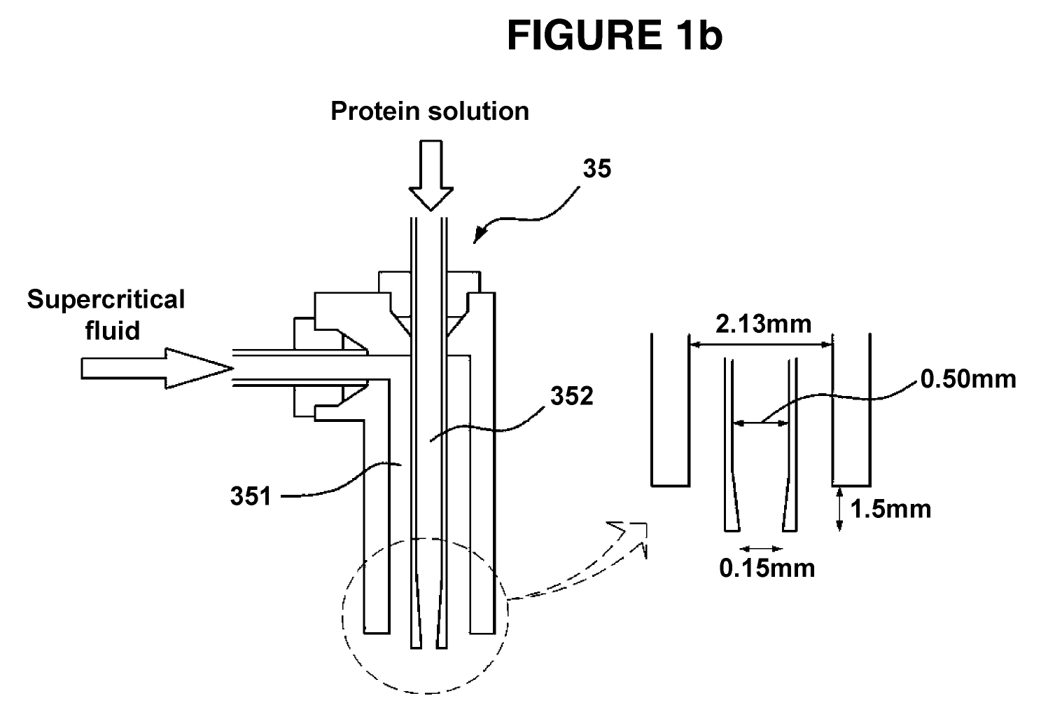

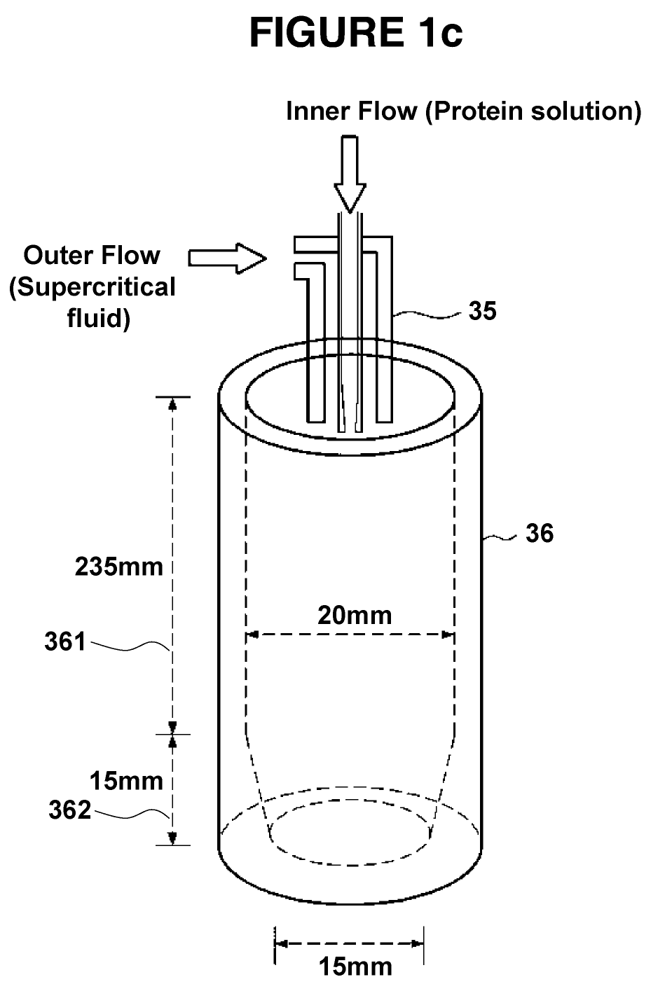

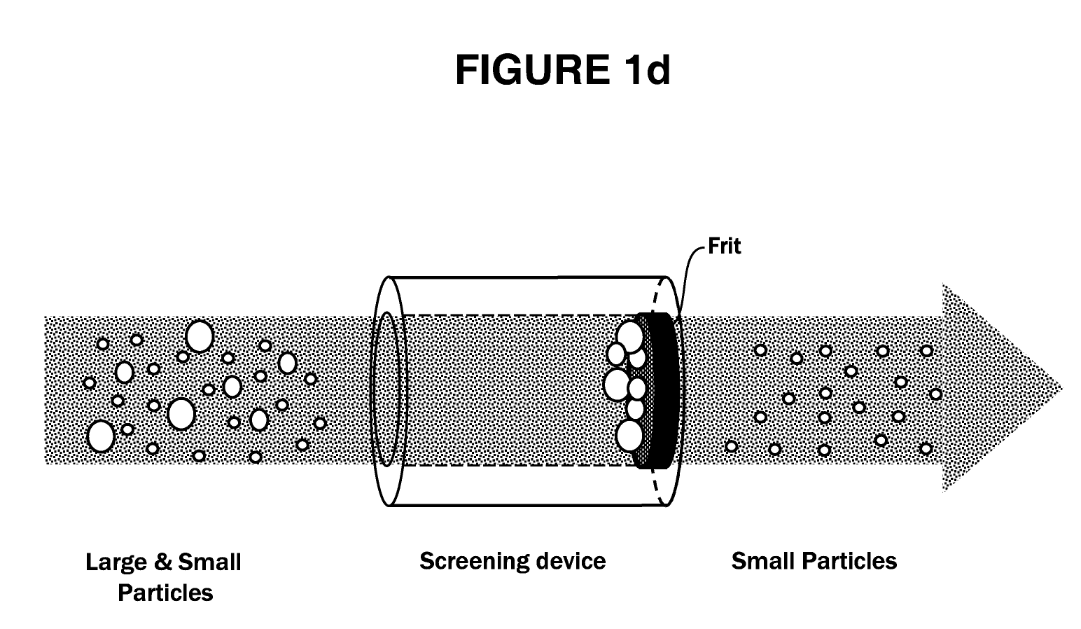

For submicronizing proteins using supercritical fluids, an apparatus for embodying optimally SEDS (solution enhanced dispersion by supercritical fluids) process was constructed. The present apparatus is schematically represented by FIG. 1a.

The present apparatus for the submicronization of proteins includes a feeding part, a precipitation part and a particle collection unit. The feeding part includes a means for feeding the supercritical fluid (1) and a means for feeding a protein solution (2). The means for feeding the supercritical fluid (1) includes a carbon dioxide cylinder (11), a cooling circulator (12) and a high pressure liquid pump (13) for moving liquid carbon dioxide (model: NS-500, Nihon Seimitsu, Japan). The carbon dioxide cylinder (11) accommodates a certain amount of carbon dioxide satisfying the requirements for temperature and pressure to maintain supercritical fluids. A valve for drawing out carbon dioxide is equipped at the outlet of the carbon dioxide cylinder. The cooling circulator (12) prevents the inhibition of the performance of the pump (13) by gaseous carbon dioxide formed by conversion from liquid carbon dioxide during passing to the pump (13). The high pressure liquid pump (13) equipped with the cooling circulator (12) allows to deliver liquid carbon dioxide with high pressure to a precipitation vessel (36).

The means for feeding a protein solution (2) are made up of a protein solution container (21) containing hGH solution and a HPLC pump (22) for pushing up the protein solution (model: SYSTEM GOLD Programmable Solvent Module 126, Beckman, USA). The HPLC pump permits the hGH solution to be sprayed with high pressure into the precipitation vessel (36).

A precipitation device (3) is made up of a device for converting liquid carbon dioxide to supercritical fluid form, and a device for inducing the oversaturation of solutes by mixing supercritical fluids and protein solutions. The precipitation device (3) includes a heat exchanger (31). The heat exchanger (31) is positioned between the high pressure liquid pump (13) and the precipitation vessel (36), allowing for the formation of supercritical fluids by heating carbon dioxide pumped with high pressure. The precipitation device (3) includes an oven (32) (JASCO CO-95) for controlling temperature by 0.1 K, a pressure gauge (33) for measuring pressure of the overall system and a back pressure regulator (34) (JASCO 880-81 BPR) for controlling the overall pressure of the system.

The device inducing the oversaturation of solutes is made up of a coaxial nozzle (35) and a precipitation vessel (36) (JASCO, Japan). The coaxial nozzle (35) was uniquely constructed by the present inventors (FIG. 1b). The outer nozzle (351) of the coaxial nozzle (35) has the inside diameter of 2.13 mm. The inner nozzle (352) of the coaxial nozzle (35) has a taper shape at its outlet end, its upper part and outlet end have the inside diameters of 0.50 mm and 0.15 mm, respectively. The outlet end of the inner nozzle is protruded by 1.5 mm toward the inner portion of the precipitation vessel compared to the outlet end of the outer nozzle.

The outlet (i.e., lower part) of the precipitation vessel (36) has a streamline shape with serially decreased diameter (FIG. 1c). Such streamline shape prevents protein particles formed from adhering and sticking to the precipitation vessel. The upper part and lower part of the precipitation vessel have the inside diameters of 20 mm and 15 mm, respectively. The upper part and lower part of the precipitation vessel have the lengths of 235 mm and 15 mm, respectively. The inside volume of the precipitation vessel is about 80 mL.

A particle collection device (4) includes a screening device (41) for filtering out larger particles and a particle collector (42) for collecting homogenous particles. The screening device (41) is equipped with a metal frit (411) having a pore size of 20 μm (FIG. 1d). The particle collector (42) is equipped with a metal frit (422) having a pore size of 0.2 μm, collecting submicronized particles formed from oversaturated solutes.

Solutions having passed through the particle collector (42) are then separated into supercritical fluids and solvents for proteins. The solvents are collected into the effluent collection device (5). The supercritical fluid having passed through particle collector (42) may be recycled or discharged into atmosphere.

### Example II

### Formation of hGH Submicroparticles Using Apparatus for Protein Submicronization

The preparation of submicroparticles using supercritical fluids is accomplished by spraying hGH solution into supercritical carbon dioxide in the precipitation vessel.

Supercritical fluids are continuously introduced into the precipitation vessel via the outer nozzle of the coaxial nozzle. The mixed solution of ethanol and a buffer (5-20 mM NaPO₄ or 5-20 mM NaPO₄/1 mM EDTA) containing protein sample (hGH) is spray-injected via the inner nozzle to form droplets with very small size.

Such spray of protein solutions in the very small-sized droplet form causes the formation of broader interface between protein solutions and supercritical fluids, to enhance mass transfer effect. At the interface formed, solvents diffuse into supercritical fluids and supercritical fluids diffuse to protein solutions, thereby rapidly inducing the oversaturation to recrystallize hGH dissolved in solutions.

hGH was dissolved in a mixed solution of ethanol and water to prepare a hGH solution with desired concentration. The temperature and pressure was adjusted to become above critical temperature of carbon dioxide and liquid carbon dioxide was introduced into the precipitation vessel (36) via the outer nozzle (351) of the coaxial nozzle (35) by use of the high pressure liquid pump (13). Upon the stabilization of the present apparatus, the hGH solution was sprayed into the precipitation vessel (36) via the inner nozzle (352) of the coaxial nozzle by use of the HPLC pump (22), thereby inducing the crystallization of hGH. After the completion of the spraying, the flow of supercritical carbon dioxide was further introduced for 1 hr to wash crystals formed by removing residual solvent in crystals. The particle collection device (4) was separated from the present apparatus and submicroparticles formed were collected. The size and distribution of submicroparticles were observed using FE-SEM.

Since the size and shape of submicroparticles finally formed depend on several parameters such as temperature, pressure and flow rate of supercritical fluids, and concentration of hGH solution, the optimal conditions for the submicronization of hGH were determined.

For the submicronization of hGH, carbon dioxide with purity of 99.9% was used as supercritical fluids, and hGH was obtained from Regeron, Inc. (South Korea). Ethanol (HPLC grade, purity 99.9%) and H₂O(HPLC grade, purity 99.9%) were commercially available from Aldrich (Milwaukee, Wis.).

### Example III

### Size Change of Submicroparticles Over Pressure of Supercritical Fluid

The density of supercritical carbon dioxide varies depending largely on its pressure. Generally, the density of supercritical carbon dioxide increases as its pressure increases. The ethanol and water in droplets sprayed undergo mass exchange with supercritical carbon dioxide. At this time, the density of supercritical carbon dioxide plays an important role in determining properties of submicroparticles formed.

The density of supercritical carbon dioxide increases as its pressure increases, which gives rise to the increase in the capacity of supercritical carbon dioxide to accommodate water and ethanol increases, thereby causing the oversaturation of solutes (hGH) in droplets in a faster manner. The rapid oversaturation generates larger amount of crystal nuclei in droplets. However, a high pressure of supercritical fluids may result in negligible change in the size of protein particulates finally formed. This is why protein droplets are sprayed into high pressurized supercritical fluids, e.g. carbon dioxide and in turn their surface area is likely to be decreased, thereby decreasing a surface area for mass exchange. In addition, the high pressure of supercritical fluids induces aggregation between protein particulates formed. Therefore, while the increase in pressure of supercritical fluids promotes the mass exchange between supercritical fluids and solvents, it also exerts the adverse effect on the surface area for mass exchange.

We varied pressures of supercritical carbon dioxide, 90, 100, 120 and 130 bar in protein submicronization and analyzed the change in the size of protein particulates using FE-SEM. The results are summarized in Table 1. If the pressure was less than 90 bar, the phase of supercritical fluids was very likely to be broken; in the case of exceeding 130 bar, the structure or conformation of proteins was liable to change.

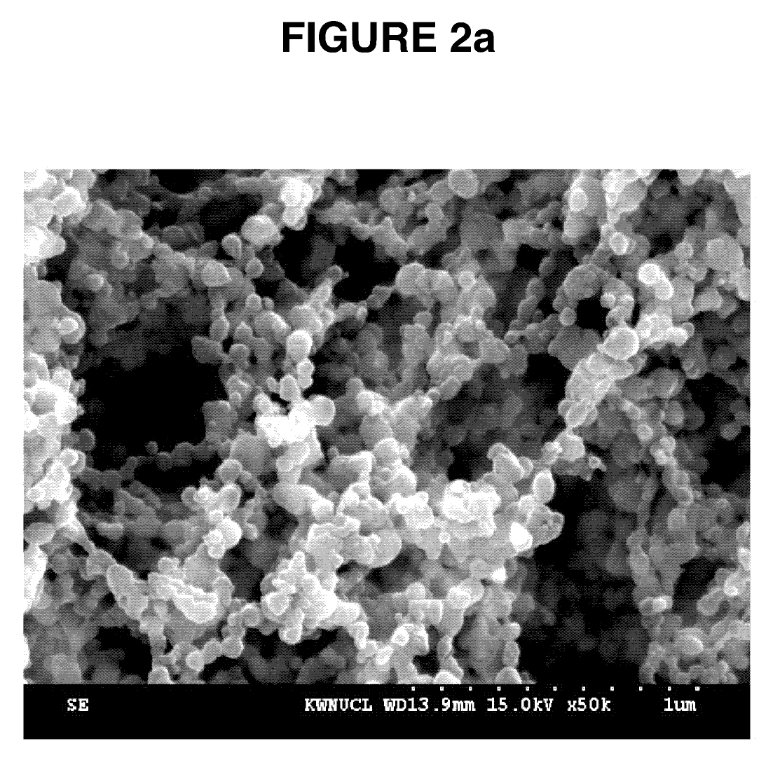

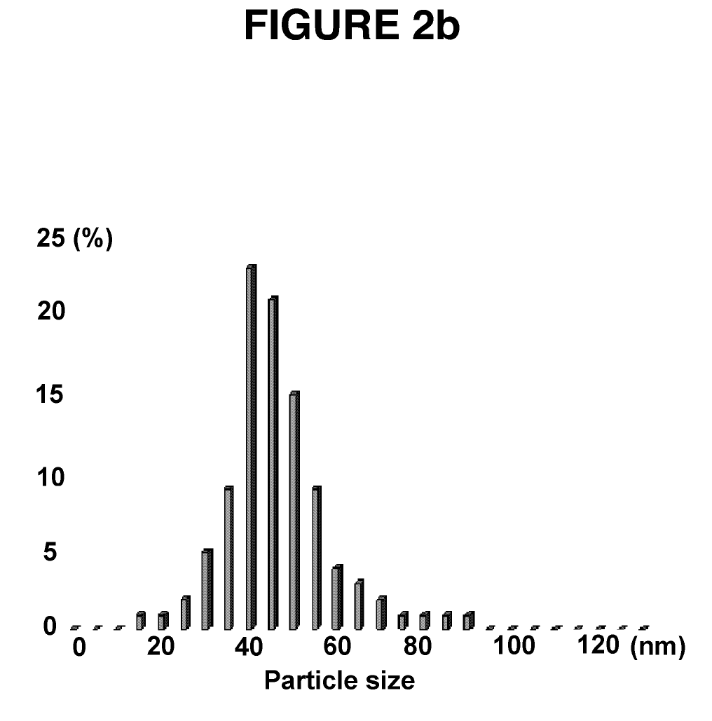

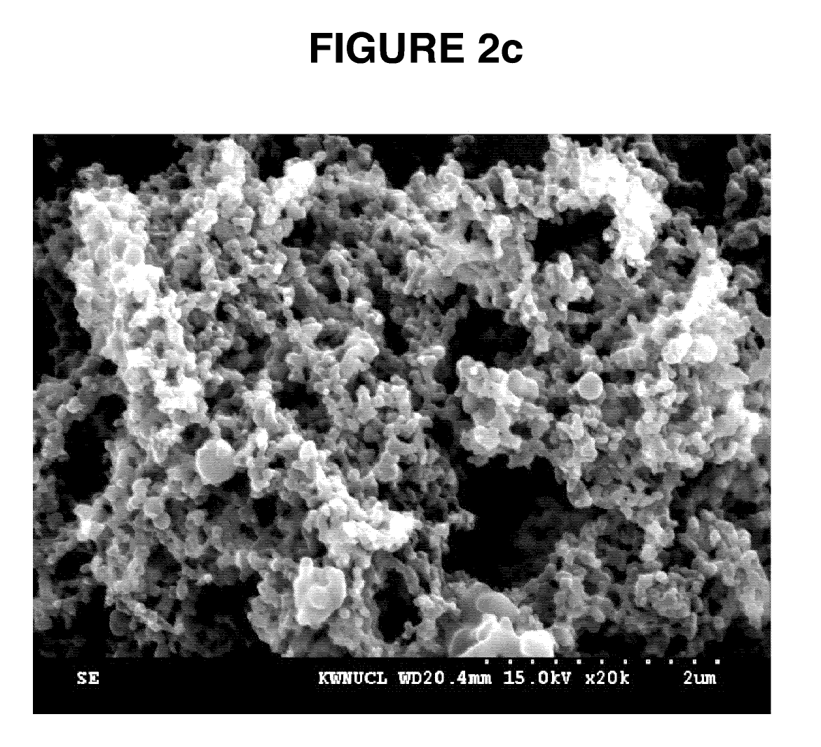

The density of supercritical carbon dioxide showed to be greatly changed as its pressure was changed around its critical pressure. Therefore, supercritical carbon dioxide with high density under lower pressure was used. As indicated in Experiment 1 of Table 1, the protein particulates with smaller size were obtained and showed narrow size distribution as shown in FIG. 2b. The results observed using FE-SEM are represented in FIG. 2a.

Under pressures going beyond critical pressure, the density change of supercritical carbon dioxide over pressure change becomes less responsive. However, it needs to be appreciated that the increase in pressure affects the decrease in surface area at lower extent than the increase in solubility. In accordance with this suggestion, the size of protein particulates increases slightly as shown in Experiment 2 and then decreases slightly as shown in Experiments 3 and 4. The FE-SEM results for Experiment 3 are shown in FIG. 2c.

TABLE 1

| | Experiment | | | |
|---|---|---|---|---|
| Parameters | 1 | 2 | 3 | 4 |
| Pressure (bar) | 90 | 100 | 120 | 130 |
| Temperature (° C.) | 40 | 40 | 40 | 40 |
| Density of CO₂ (kg/m³) | 486 | 629 | 718 | 743 |
| Flow rate of CO₂ (ml/min) | 30 | 30 | 30 | 30 |
| Flow rate of protein solution (ml/min) | 0.3 | 0.3 | 0.3 | 0.3 |
| Concentration of hGH (mg/L) | 24.4 | 24.4 | 24.4 | 24.4 |
| Component ratio of solvent | 20:1 | 20:1 | 20:1 | 20:1 |
| (ethanol:water (v/v)) | | | | |
| Mean particle diameter (nm) | 45 | 57 | 53 | 50 |

### Example IV

### Size Change of Submicroparticles Over Temperature of Supercritical Fluid

The protein submicroparticles were prepared with changing temperature of supercritical carbon dioxide, 40° C., 50° C., 60° C. and 70° C. The size of submicroparticles was analyzed using FE-SEM and summarized in Table 2.

The temperature of supercritical carbon dioxide influences greatly on the size of particles formed. The surface tension of sprayed droplets of protein solutions is inversely proportional to temperature. The increase in temperature induces the decrease in the surface tension of droplets, which makes it easier for supercritical carbon dioxide to penetrate into droplets. Then, mass exchange between ethanol and water in droplets and supercritical carbon dioxide may be more active.

However, extremely high temperature is very likely to elicit aggregation between protein particulates. Furthermore, the increase in temperature under certain pressure results in the decrease in the density of supercritical carbon dioxide, to reduce the rate of oversaturation of a solute (hGH) in droplets, finally forming lager protein particulates.

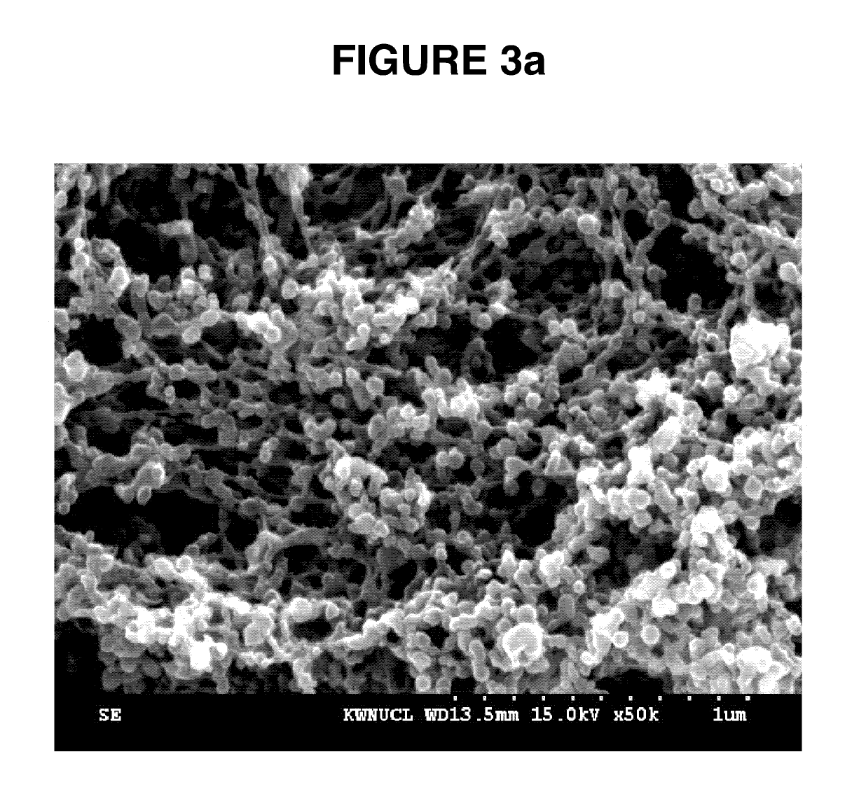

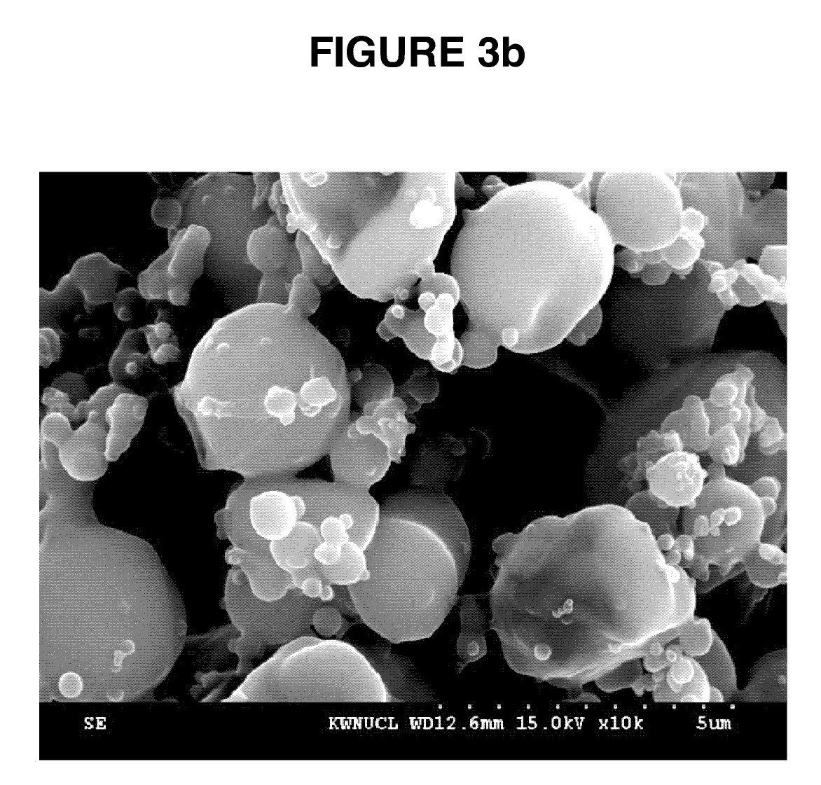

The FE-SEM image (FIG. 3a) of Experiment 6 in Table 2 shows no aggregation, whereas that (FIG. 3b) of Experiment 8 in Table 2 shows aggregation of protein particulates.

TABLE 2

| | Experiment | | | |
|---|---|---|---|---|
| Parameters | 5 | 6 | 7 | 8 |
| Pressure (bar) | 100 | 100 | 100 | 100 |
| Temperature (° C.) | 35 | 40 | 50 | 60 |
| Density of CO₂ (kg/m³) | 712 | 629 | 384 | 289 |
| Flow rate of CO₂ (ml/min) | 30 | 30 | 30 | 30 |
| Flow rate of protein solution (ml/min) | 0.3 | 0.3 | 0.3 | 0.3 |
| Concentration of hGH (mg/L) | 24.4 | 24.4 | 24.4 | 24.4 |
| Component ratio of solvent | 25:1 | 25:1 | 25:1 | 25:1 |
| (ethanol:water (v/v)) | | | | |
| Mean particle diameter (nm) | 60 | 45 | 140 | 980 |

### Example V

### Size Change of Submicroparticles Over Concentration of Protein Solution

The concentration of protein solution is one of the significant factors in the formation of hGH submicroparticles using supercritical fluids.

hGH in droplets forms crystal nuclei during mass exchange between ethanol and water in protein solution and supercritical carbon dioxide. At this time, if the concentration of the hGH solution is low, the formation of crystal nuclei occurs slowly and hGH is aggregated around the crystal nuclei to produce larger particulates. In contrast, where the concentration of the hGH solution is high, numerous crystal nuclei are formed in a more rapid manner to be bound each other, thereby decreasing the number of crystal nuclei formed earlier, thus increasing the size of final protein particulates.

The protein submicroparticles were prepared with changing the concentration of protein solution, 12.2 mg/L, 24.4 mg/L, 48.8 mg/L and 97.6 mg/L. The size of submicroparticles was analyzed using FE-SEM and summarized in Table 3.

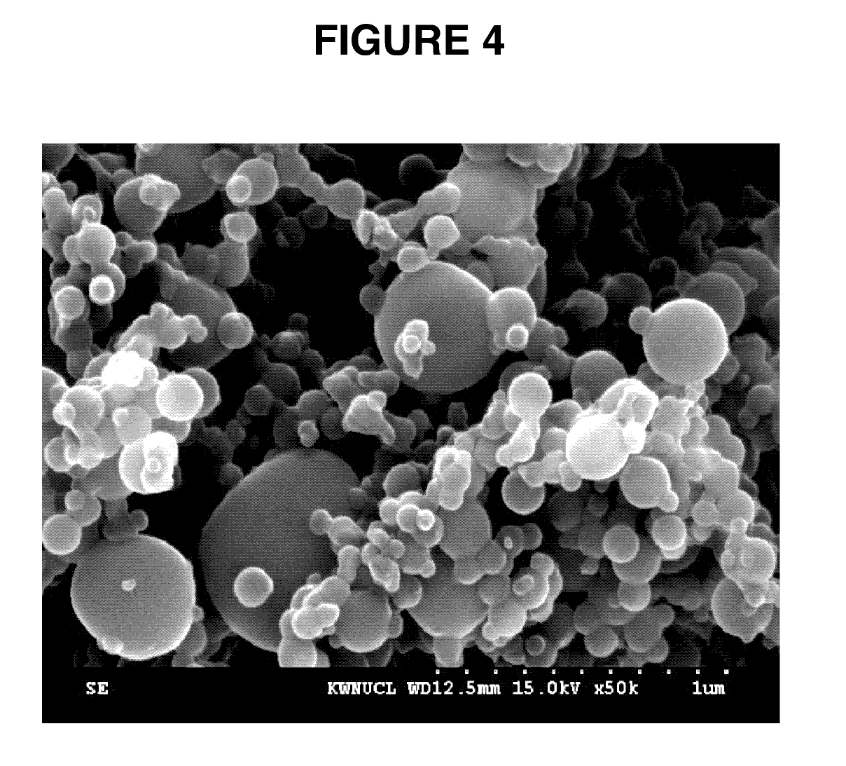

The size of the hGH submicroparticles decreases as the concentration of hGH solution increases from 12.2 mg/L to 24.4 mg/L. Where the concentration of hGH solution elevates above 24.4 mg/L, the size of hGH submicroparticles increases. These results demonstrate that 24.4 mg/L of hGH solution is optimal concentration to form crystal nucleus at the most preferable rate. FIG. 4 represents FE-SEM analysis results of Experiment 10 with 24.4 mg/L of hGH solution

TABLE 3

| | Experiment | | | |
|---|---|---|---|---|
| Parameters | 9 | 10 | 11 | 12 |
| Pressure (bar) | 100 | 100 | 100 | 100 |
| Temperature (° C.) | 40 | 40 | 40 | 60 |
| Density of CO₂ (kg/m³) | 629 | 629 | 629 | 629 |
| Flow rate of CO₂ (ml/min) | 30 | 30 | 30 | 30 |
| Flow rate of protein solution (ml/min) | 0.3 | 0.3 | 0.3 | 0.3 |
| Concentration of hGH (mg/L) | 12.2 | 24.4 | 48.8 | 97.6 |
| Component ratio of solvent | 25:1 | 25:1 | 25:1 | 25:1 |
| (ethanol:water (v/v)) | | | | |
| Mean particle diameter (nm) | 140 | 45 | 980 | 1850 |

### Example VI

### Analysis of hGH Submicroparticles

The hGH submicroparticles prepared in the above Examples were analyzed to evaluate their activity and the occurrence of denaturation. The powder of hGH submicroparticles was resuspended in 30 mM NaPO₄ (pH 8.0) and then analyzed using HPLC (Shimadzu, 0.1% TFA in acetonitrile:0.1% TFA in H₂O, C-18 Waters column).

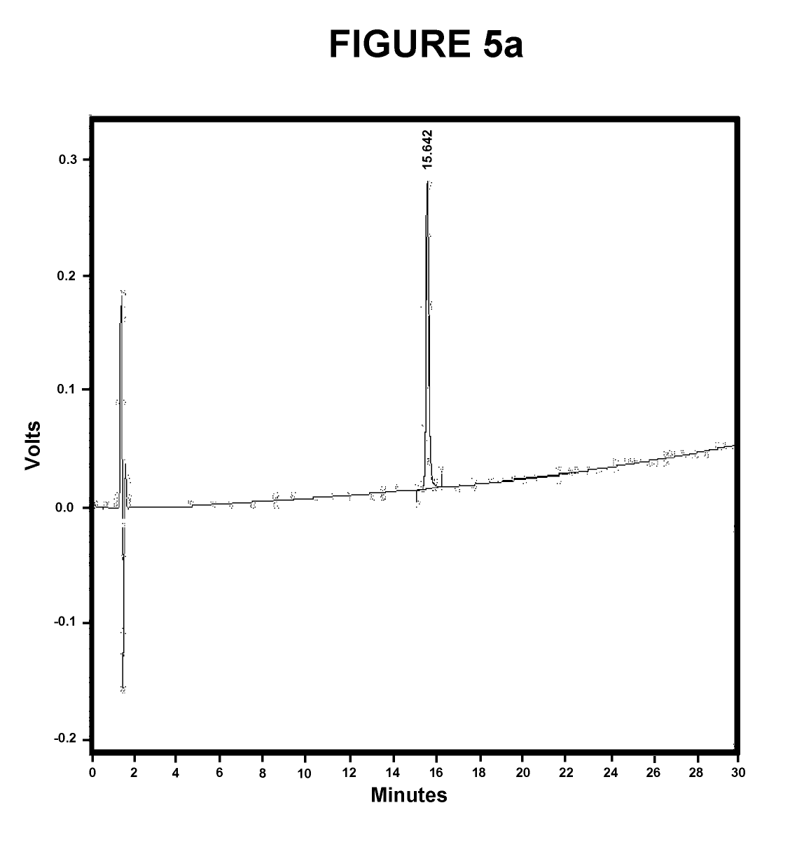

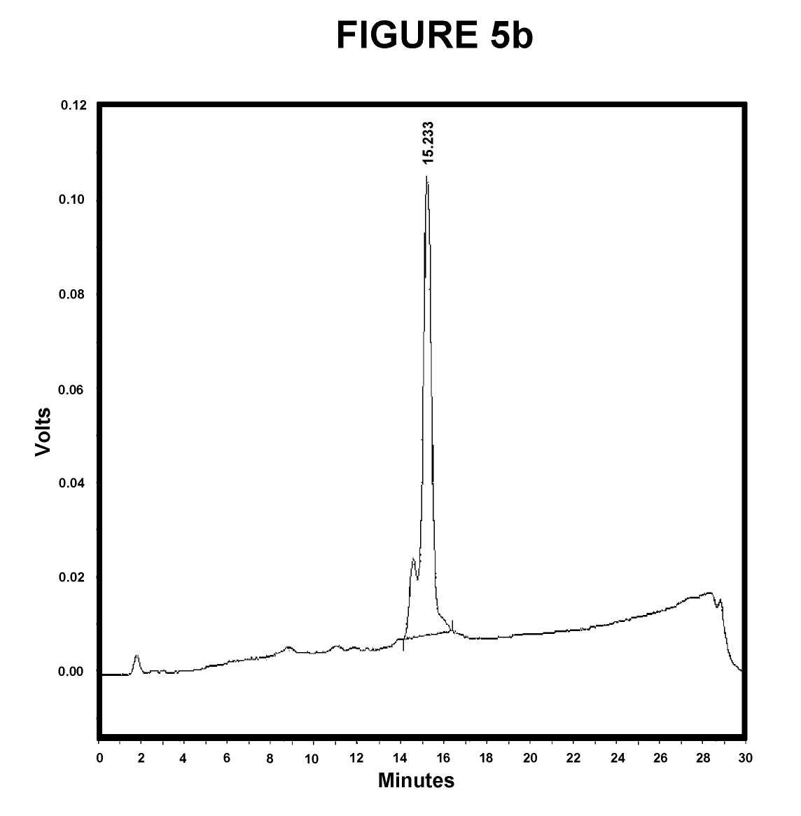

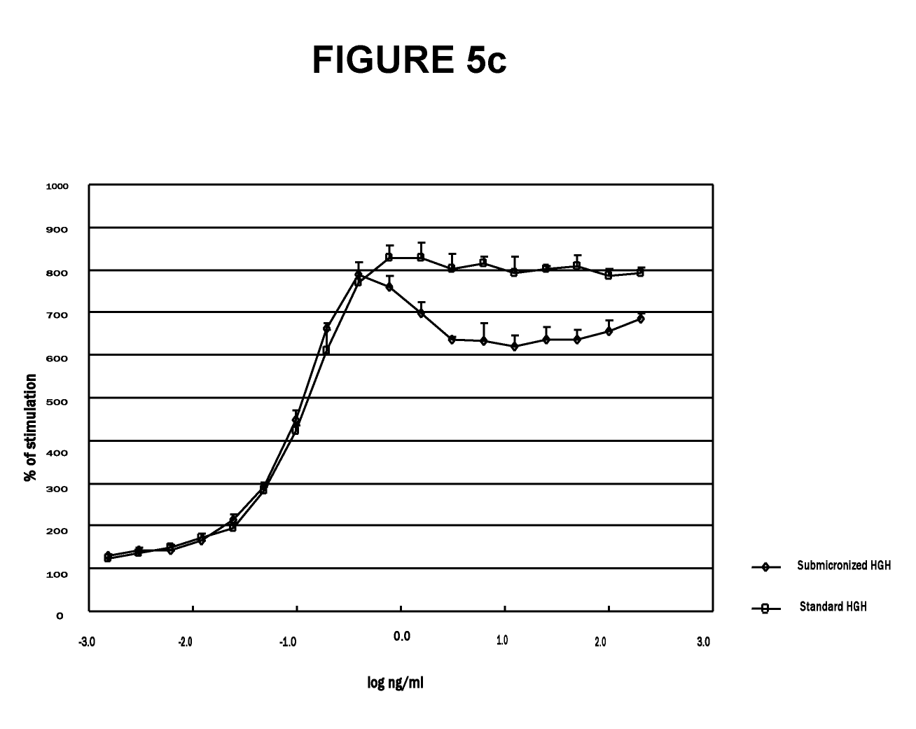

As shown in FIGS. 5a-5b, most of hGH molecules in submicroparticles formed according to the present invention were not denatured, which can be verified by comparing peaks around retention time of 15.5 min corresponding to NIBSC standard hGH and hGH of this invention. The small peak appearing before the hGH peak of this invention could be recognized as deamidated hGH formed during storage of hGH sample solutions

The activity of hGH submicroparticles formed according to this invention was measured using mouse Nb2 cell line. To the wells of a 96-well plate containing 50 μl of the Nb₂ noble rat lymphoma cell line (NIBSC ECACC #97041101) at a density of 1×10⁵ cells/ml, added to standard hGH (NIBSC code 98/574) and hGH submicroparticles of this invention (sample hGH). After the incubation under 5% CO₂ for 5 days at 37° C., the amount of proliferated cells was measured using MTT.

As shown in FIG. 5c, hGH of this invention exhibits the activity corresponding to above 95% of standard hGH activity, demonstrating hGH submicroparticles of this invention possess almost the inherent full activity of hGH.

Meanwhile, the solubility and biological activity of hGH submicroparticles formed according to this invention are greatly affected by their particle size, which is summarized in Table 4. In Table 4, the conditions for Experiment 13 are 100 bar, 40° C., 30 ml/min of carbon dioxide flow rate, 0.3 ml/min of protein solution flow rate and 0.018 mg/ml of protein solution concentration, those for Experiment 14, 100 bar, 40° C., 30 ml/min of carbon dioxide flow rate, 0.3 ml/min of protein solution flow rate and 0.024 mg/ml of protein solution concentration, and those for Experiment 15, 100 bar, 40° C., 40 ml/min of carbon dioxide flow rate, 0.5 ml/min of protein solution flow rate and 0.018 mg/ml of protein solution concentration.

The solubility and activity of hGH submicroparticles are shown to be higher as their particle size decreases. In particular, 97.7% of the biological activity was exhibited for submicroparticles with 55 nm size.

TABLE 4

| | | Conc. for | Conc. for | | |
|---|---|---|---|---|---|
| | | maximum | maximum | | |
| | Mean | activity | activity (hGH | | |
| | particle | (hGH | submicroparticls | | Biological |
| | diameter | standard | of this | Solubility | activity |
| Exp. | (nm) | solution) | invention) | (%) | (%) |
| Exp. | 55 | 1.5 ng/mL | 1.6 ng/mL | 100 | 97.7 |
| 13 | | | | | |
| Exp. | 100 | 1.5 ng/mL | 3.0 ng/mL | 60 | 50.0 |
| 14 | | | | | |
| Exp. | 200 | 1.5 ng/mL | 11.8 ng/mL | 10 | 12.6 |
| 15 | | | | | |

As described hereinabove, the present invention provides apparatuses for the submicronization of proteins. In addition, the present invention provides methods for the submicronization of proteins.

The present invention provides more homogeneous and smaller protein (particularly, human growth hormone) particulates, permitting the production of transpulmonary drugs of proteins (particularly, human growth hormone). Moreover, the present invention ensure that submicronized protein powder possessing its inherent activities can be prepared with higher concentration in smaller volume, by no use of diluents and vehicles generally used with the amount of 15-fold larger than protein active ingredients to prepare protein (particularly, human growth hormone) powder for injection. Therefore, the present invention allows for the development of protein drugs for oral administration. In addition to this, the present invention provides the powder of human growth hormone including nanoscale particulates and enables reduction of the moisture content to less than 0.3%. The moisture content of the conventional freeze-dried powder is in the range of 0.3-10%. In this context, the powder of human growth hormone prepared according to this invention is very advantageous in view of long term storage.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. An apparatus for the submicronization of a protein using a supercritical fluid, which comprises:
means for feeding the supercritical fluid;
means for feeding a protein solution;
a precipitation vessel with a taper shape at its lower part, wherein the precipitation vessel accommodates the supercritical fluid and the protein solution to generate submicroparticles of the protein; and
a spray nozzle with coaxial arrangement comprising an outer nozzle for spraying the supercritical fluid and an inner nozzle for spraying the protein solution, wherein the spray nozzle is connected to the means for feeding the supercritical fluid and the means for feeding a protein solution; wherein an outlet end of the inner nozzle is more protruded than an outlet end of the outer nozzle toward an inner portion of the precipitation vessel; and wherein an contact between the supercritical fluid and the protein solution occurs in the precipitation vessel.

2. The apparatus according to claim 1, wherein the protein is selected from the group consisting of hormone, hormone analogue, enzyme, enzyme inhibitor, signal transduction protein or fragment thereof, antibody or fragment thereof, single chain antibody, binding protein or fragment thereof, peptide, antigen, adhesive protein, structural protein, regulatory protein, toxin protein, cytokine, transcription regulatory protein, blood clotting protein and plant defense-inducing protein.

3. The apparatus according to claim 2, wherein the protein is human growth hormone.

4. The apparatus according to claim 1, wherein the supercritical fluid is selected from the group consisting of carbon dioxide, ethane, ethylene, sulfur hexafluoride, nitrous oxide, chlorotrifluoromethane, monofluoromethane, xenone and combinations thereof.

5. The apparatus according to claim 4, wherein the supercritical fluid is carbon dioxide.

6. The apparatus according to claim 1, wherein the protein solution is prepared by dissolving the protein into a solvent selected from the group consisting of water, ethanol, methanol, DMSO, isopropanol, acetone, THF, acetic acid, ethyleneglycol, polyethyleneglycol, N,N-dimethylaniline and combinations thereof.

7. The apparatus according to claim 6, wherein the protein solution is prepared by dissolving the protein into a mixture of water and ethanol.

8. The apparatus according to claim 1, wherein an upper part of the precipitation vessel has a diameter of 1.2-1.5 fold larger than the end of the lower part of the precipitation vessel.

9. The apparatus according to claim 1, wherein an upper part of the precipitation vessel has a length of 10-18 fold longer than the lower part of the precipitation vessel.

10. The apparatus according to claim 1, wherein the outer nozzle has a diameter of 3-6 fold larger than the inner nozzle.

11. The apparatus according to claim 1, wherein the outer nozzle has a substantially constant diameter along its overall length.

12. The apparatus according to claim 1, wherein the inner nozzle has a taper shape at its outlet end.

13. The apparatus according to claim 12, wherein an upper part of the inner nozzle has a diameter of 2-4 fold larger than the outlet end of the inner nozzle.

14. The apparatus according to claim 1, wherein the outlet end of the inner nozzle is protruded by 1-3 mm toward the inner portion of the precipitation vessel compared to the outlet end of the outer nozzle.

15. The apparatus according to claim 1, wherein the apparatus further comprises a screening device having a filter with a pore size of 5-40 μm and connected to the precipitation vessel.

16. The apparatus according to claim 5, wherein the supercritical fluid has a pressure of 90-130 bar.

17. The apparatus according to claim 16, wherein the supercritical fluid has a pressure of 90-100 bar.

18. The apparatus according to claim 5, wherein a temperature in the precipitation vessel maintains within 38-45° C.

19. The apparatus according to claim 18, wherein the temperature in the precipitation vessel maintains around 40° C.

20. The apparatus according to claim 1, wherein the protein solution has a concentration of 10-300 mg/L.

21. The apparatus according to claim 20, wherein the protein solution has a concentration of 20-150 mg/L.

22. The apparatus according to claim 1, wherein the ratio of a flow rate of the supercritical fluid fed by the means for feeding the supercritical fluid to a flow rate of the protein solution fed by the means for feeding the protein solution ranges from 50:1 to 120:1.

23. The apparatus according to claim 22, wherein the ratio of the flow rate of the supercritical fluid fed by the means for feeding the supercritical fluid to the flow rate of the protein solution fed by the means for feeding the protein solution ranges from 75:1 to 110:1.

24. The apparatus according to claim 1, wherein a flow rate of the supercritical fluid fed by the means for feeding the supercritical fluid is 10-40 ml/min.

25. The apparatus according to claim 24, wherein the flow rate of the supercritical fluid fed by the means for feeding the supercritical fluid is 20-30 ml/min.

26. The apparatus according to claim 1, wherein a flow rate of the protein solution fed by the means for feeding the protein solution is 0.2-0.8 ml/min.

27. The apparatus according to claim 26, wherein the flow rate of the protein solution fed by the means for feeding the protein solution is 0.3-0.5 ml/min.

28. The apparatus according to claim 1, wherein the submicroparticles of the protein generated by the apparatus have an average diameter of 30-60 nm.

29. The apparatus according to claim 1, wherein the submicroparticles of the protein generated by the apparatus have a size distribution of no less than 90% within an average diameter of 35-55 nm.

30. The apparatus according to claim 15, wherein the apparatus further comprises a particle collector having a filter with a pore size of 0.1-1 μm and connected to the screening device.

31. A method for the submicronization of a protein, comprising the steps of:
(i) mixing a protein solution and a supercritical fluid by spray injecting the protein solution and the supercritical fluid into an inner portion of a precipitation vessel through a spray nozzle with coaxial arrangement; and
(ii) obtaining submicronized protein particles generated by the precipitation from the protein solution,
wherein the supercritical fluid is carbon dioxide, the supercritical fluid has a pressure of 90-130 bar, a temperature in the precipitation vessel is maintained within 35-45° C. and the protein solution has a concentration of 10-300 mg/L.

32. The method according to claim 31, wherein the protein is selected from the group consisting of hormone, hormone analogue, enzyme, enzyme inhibitor, signal transduction protein or fragment thereof, antibody or fragment thereof, single chain antibody, binding protein or fragment thereof, peptide, antigen, adhesive protein, structural protein, regulatory protein, toxin protein, cytokine, transcription regulatory protein, blood clotting protein and plant defense-inducing protein.

33. The method according to claim 32, wherein the protein is human growth hormone.

34. The method according to claim 31, wherein the protein solution is prepared by dissolving the protein into a solvent selected from the group consisting of water, ethanol, methanol, DMSO, isopropanol, acetone, THF, acetic acid, ethyleneglycol, polyethyleneglycol, N,N-dimethylaniline and combinations thereof.

35. The method according to claim 34, wherein the protein solution is prepared by dissolving the protein into a mixture of water and ethanol.

36. The method according to claim 31, wherein the supercritical fluid has a pressure of 90-100 bar.

37. The method according to claim 31, wherein the temperature in the precipitation vessel maintains around 40° C.

38. The method according to claim 31, wherein the protein solution has a concentration of 20-150 mg/L.

39. The method according to claim 31, wherein the ratio of a flow rate of the supercritical fluid to a flow rate of the protein solution ranges from 50:1 to 120:1.

40. The method according to claim 39, wherein the ratio of the flow rate of the supercritical fluid to the flow rate of the protein solution ranges from 75:1 to 110:1.

41. The method according to claim 31, wherein a flow rate of the supercritical fluid is 10-40 ml/min.

42. The method according to claim 41, wherein the flow rate of the supercritical fluid is 20-30 ml/min.

43. The method according to claim 31, wherein a flow rate of the protein solution is 0.2-0.8 ml/min.

44. The method according to claim 43, wherein the flow rate of the protein solution is 0.3-0.5 ml/min.

45. The method according to claim 31, wherein the submicronized protein particles have an average diameter of 30-60 nm.

46. The method according to claim 31, wherein the submicronized protein particles have a size distribution of no less than 90% within an average diameter of 35-55 nm.

47. The method according to claim 31, wherein the protein solution and the supercritical fluid in step (i) are spray-injected through an inner nozzle and an outer nozzle of a spray nozzle with coaxial arrangement, respectively, and an outlet end of the inner nozzle is more protruded than an outlet end of the outer nozzle toward the inner portion of the precipitation vessel.
